(19) United States
(12) Patent Application Publication
(10) Pub. No.: US 2026/0140026 A1
(43) Pub. Date: May 21, 2026

(54) INTELLIGENT DEDUCTION METHOD OF DATA-PHYSICAL FUSION OF PORE TOPOLOGY CONFIGURATION OF DEEP ROCK MASS BASED ON EXPERIMENTS

(71) Applicant: Tongji University, Shanghai (CN)

(72) Inventors: Xilin LYU, Shanghai (CN); Yu ZENG, Shanghai (CN); Dawei XUE, Shanghai (CN); Xianlin LIU, Nanning (CN); Yu SHAO, Nanning (CN); Yixin LU, Nanning (CN)

(21) Appl. No.: 19/019,262

(22) Filed: Jan. 13, 2025

(30) Foreign Application Priority Data
Nov. 15, 2024 (CN) 202411641269.8

(51) Int. Cl. G01N 3/10; G01N 3/06; G01N 33/24; G06T 7/00; G06V 10/40; G06V 10/77; G06V 10/82

(52) U.S. Cl. CPC G01N 3/10; G01N 3/068; G01N 33/24; G06T 7/0002; G06V 10/40; G06V 10/7715; G06V 10/82; G06T 2207/10028; G06T 2207/20084; G06T 2207/30181; G06T 2207/30204

(57) ABSTRACT

An intelligent deduction method of data-physical fusion of pore topology configuration of deep rock mass based on experiments based on experiments is provided, including: obtaining a standard rock sample of a deep rock mass; marking the standard rock sample to obtain a test standard rock sample; carrying out a loaded catastrophe test on the test standard rock sample to obtain a mining process and test data of the deep rock mass; obtaining point cloud information of a loading process of the test standard rock sample by a physical method; carrying out a three-dimensional reconstruction on the test standard rock sample by a mathematical method according to the point cloud data to obtain a spatial dynamic evolution of a loaded behavior of the deep rock mass; and carrying out a feature extraction on the pore topology configuration of a deep loaded rock mass to obtain dynamic evolution features.

### CROSS-REFERENCE TO RELATED APPLICATIONS

This application claims priority to Chinese Patent Application No. 202411641269.8, filed on Nov. 15, 2024, the contents of which are hereby incorporated by reference.

### TECHNICAL FIELD

The disclosure belongs to the technical field of pore deduction of deep rock mass, and in particular to an intelligent deduction method of data-physical fusion of pore topology configuration of deep rock mass based on experiments.

### BACKGROUND

With the increasing demand for energy and the gradual depletion of shallow resources, energy development is gradually moving towards the deep part of the earth. Deep rock mass has been under load for a long time in a complex environment, and it is easy to induce deep engineering disasters once it is improperly controlled during mining. Pore topology configuration is an important index to describe the physical mechanics and permeability features of rock. By studying the evolution of pore topology configuration, the mechanical response and failure mechanism of rock mass during excavation may be predicted more accurately, and the deep energy storage and migration conditions may be evaluated to guide the exploration and development of deep energy.

Restricted by the complex environment of deep rock mass, the invisibility of the interior of rock mass and the complex pore topology configuration, the distribution of pores in deep rock mass is not easy to obtain. At present, the determination of pores in rock mass is mainly concentrated in laboratory tests. Although there are a variety of related testing methods, most are pore measurements in a single state of rock mass, which may not realize the measurement of the whole process of rock mass from initial conditions to loading failure. The pore topology configuration of rock mass is randomly distributed in space and complex and changeable, so the existing technology may not accurately obtain the dynamic evolution process of pore topology configuration of rock mass. In addition, due to the greater difficulty in obtaining and limited number of deep rock samples, the sample data obtained by repeated tests are small, and the limited test samples are difficult to accurately reflect the real evolution law of pore topology configuration of deep loaded rock mass.

### SUMMARY

In order to solve the above technical problems, the disclosure provides an intelligent deduction method of data-physical fusion of pore topology configuration of deep rock mass based on experiments, so as to solve the problems existing in the prior art.

In order to achieve the above objectives, in the first aspect, the disclosure provides an intelligent deduction method of data-physical fusion of pore topology configuration of deep rock mass based on experiments, including the following steps:
obtaining a standard rock sample of a deep rock mass;
marking the standard rock sample to obtain a test standard rock sample;
carrying out a loaded catastrophe test on the test standard rock sample to obtain a mining process and test data of the deep rock mass;
according to the mining process and the test data of the deep rock mass, obtaining point cloud information of a loading process of the test standard rock sample by a physical method;
carrying out a three-dimensional reconstruction on the test standard rock sample by a mathematical method according to the point cloud data to obtain a spatial dynamic evolution of a loaded behavior of the deep rock mass; and
carrying out a feature extraction on the pore topology configuration of a deep loaded rock mass to obtain dynamic evolution features according to the spatial dynamic evolution.

Optionally, marking the standard rock sample includes:
using an isotope vacuum saturation tracer device to mark the standard rock sample with C-14; where the isotope vacuum saturation tracer device includes a vacuum pump, a valve A, a saturation cylinder, the standard rock sample, a pressure gauge, a valve B and a water injection tank.

Optionally, carrying out the loaded catastrophe test on the test standard rock sample includes:
carrying out the loaded catastrophe test on the test standard rock sample by adopting a dynamic evolution tracking loading device; the dynamic evolution tracking loading device includes a supporting base, a first horizontal loading device, a second horizontal loading device, a circular guide rail, a vertical loading device and a vertical loading oil cylinder.

Optionally, obtaining the point cloud information of the loading process of the test standard rock sample includes:
according to the mining process and the test data of the deep rock mass, obtaining a spatial distance between the test standard rock sample and an isotope detecting and tracking device;
calculating spatial coordinates of the test standard rock sample according to the spatial distance; and
according to the spatial coordinates, obtaining spatial point cloud data of the loading process of the test standard rock sample.

Optionally, carrying out the feature extraction on the pore topology configuration of the deep loaded rock mass including:
obtaining a two-dimensional sample image of the test standard rock sample according to the spatial dynamic evolution; and
using a fully convolutional neural network to carry out the feature extraction of the pore topology configuration on the two-dimensional sample image to obtain the dynamic evolution features.

In a second aspect, the disclosure also discloses an intelligent deduction system of data-physical fusion of pore topology configuration of deep rock mass based on experiments, where the system includes:
a rock sample acquisition module, used for obtaining a standard rock sample of a deep rock mass;
a rock sample marking module, used for marking the standard rock sample to obtain a test standard rock sample;
a rock sample test module, used for carrying out a loaded catastrophe test on the test standard rock sample to obtain a mining process and test data of the deep rock mass;
a three-dimensional reconstruction module, used for obtaining point cloud information of a loading process of the test standard rock sample according to the mining process and the test data of the deep rock mass; carrying out a three-dimensional reconstruction on the test standard rock sample according to point cloud data to obtain a spatial dynamic evolution of a loaded behavior of the deep rock mass; and
a feature extraction module, used for carrying out a feature extraction on the pore topology configuration of a deep loaded rock mass to obtain dynamic evolution features according to the spatial dynamic evolution.

In a third aspect, the disclosure also discloses a computer device, including a memory, a processor and a computer program stored on the memory, where the processor executes the computer program to realize the steps of the method of the first aspect.

In a fourth aspect, the disclosure also discloses a computer-readable storage medium, on which a computer program is stored, where when the computer program is executed by a processor, steps of the method of the first aspect are realized.

In a fifth aspect, the disclosure also discloses a computer program product, including a computer program, where when the computer program is executed by a processor, the steps of the method of the first aspect are realized.

Compared with the prior art, the disclosure has the following advantages and technical effects.

The disclosure provides an intelligent deduction method of data-physical fusion of pore topology configuration of deep rock mass based on experiments based on experiments, which includes the following steps: obtaining a standard rock sample of a deep rock mass; marking the standard rock sample to obtain a test standard rock sample; carrying out a loaded catastrophe test on the test standard rock sample to obtain a mining process and test data of the deep rock mass; according to the mining process and the test data of the deep rock mass, obtaining point cloud information of a loading process of the test standard rock sample by a physical method; carrying out a three-dimensional reconstruction on the test standard rock sample by a mathematical method according to the point cloud data to obtain a spatial dynamic evolution of a loaded behavior of the deep rock mass; and carrying out a feature extraction on the pore topology configuration of a deep loaded rock mass to obtain dynamic evolution features according to the spatial dynamic evolution.

According to the disclosure, the experimental testing technology is combined with an intelligent algorithm, and based on the data-physical fusion technology, the real-time capture of the loaded catastrophe process of deep rock mass and the point cloud information is realized, and the spatial dynamic evolution of the loaded behavior of deep rock mass is reproduced through the three-dimensional reconstruction of the point cloud data, so that the acquisition and intelligent prediction of the dynamic evolution features of the pore topology configuration of deep loaded rock mass are realized. Compare with that prior art, the disclosure overcomes the problem of dynamic observation of the pore topology configuration of the deep loaded rock mass, reflects the real evolution law of the pore topology configuration of the deep rock mass through a limited number of indoor unit tests, and is of great significance for revealing the mining disaster and stability evaluation of the deep energy-bearing rock mass.

### BRIEF DESCRIPTION OF THE DRAWINGS

The accompanying drawings, which constitute a part of this application, are used to provide a further understanding of this application. The illustrative embodiments of this application and their descriptions are used to explain this application, and do not constitute an improper limitation of this application. In the attached drawings:

FIG. 1 is a schematic diagram of in-situ loaded environment and sampling processing of deep rock mass according to an embodiment of the present disclosure.

FIG. 2 is a schematic diagram of an isotope vacuum saturation tracer device according to an embodiment of the present disclosure.

FIG. 3 is a schematic diagram of the main structure of the dynamic evolution tracking loading device of pore topology configuration under the loaded condition of deep rock mass according to an embodiment of the present disclosure.

FIG. 4 is a three-view diagram of the dynamic evolution tracking loading device of pore topology configuration under the loaded condition of deep rock mass according to an embodiment of the present disclosure.

FIG. 5 is a schematic diagram of the structural composition of the dynamic evolution tracking loading device of pore topology configuration under the loaded condition of deep rock mass according to an embodiment of the present disclosure.

FIG. 6 is a schematic structural diagram of an isotope detecting and tracking device according to an embodiment of the present disclosure.

FIG. 7 is a schematic diagram of dynamic tracking of spatial deformation point cloud data of loaded rock mass according to an embodiment of the present disclosure.

FIG. 8 is a flowchart of three-dimensional reconstruction of spatial point cloud information of loaded rock mass according to an embodiment of the present disclosure.

FIG. 9 is a flow chart for realizing spatial evolution intelligent deduction of pore topology configuration feature extraction based on fully convolutional neural network according to an embodiment of the present disclosure.

FIG. 10 is a flowchart of a method according to an embodiment of the present disclosure.

### DETAILED DESCRIPTION OF THE EMBODIMENTS

It should be noted that the embodiments in this application and the features in the embodiments may be combined with each other without conflict. The present application will be described in detail with reference to the attached drawings and examples.

It should be noted that the steps shown in the flowchart of the accompanying drawings may be executed in a computer system such as a set of computer-executable instructions, and although the logical order is shown in the flowchart, in some cases, the steps shown or described may be executed in a different order from here.

Pore topology configuration refers to a mathematical structure that studies the spatial shape and deformation of pore structure without considering the specific measurement and distance. It pays attention to the connectivity, adjacency and continuity of pores, but does not pay attention to the specific measurement and distance in space.

Pore topology has an important application in materials science, especially in the study of porous materials. Porous materials are basically a grid-like structure composed of crossed pillars and plates, and these compartments are packed and assembled together to fill the space. The mechanical properties of porous materials may be determined by their composition, structure and relative density, which is defined by the ratio of the density of porous materials to the solid density of the same materials. Topological configuration of porous materials has a significant influence on mechanical properties. For example, the microstructure of open-cell foam consists of a grid arrangement of interconnected pillars in three-dimensional space, while closed-cell foam contains a plate-like surface with a certain thickness and length. These structures not only affect the mechanical properties of materials, but also determine their performance in practical applications.

It is of great significance to study the pore topology configuration for understanding the basic physical significance of unique effects in nano-materials. By understanding these topological structures, we may better design and manufacture materials with specific properties, so as to synthesize metamaterials with adjustable mechanical properties on various length scales.

In this embodiment, an intelligent deduction method of data-physical fusion of pore topology configuration of deep rock mass based on experiments is provided, which includes the following steps:

S1, obtaining a standard rock sample of a deep rock mass;

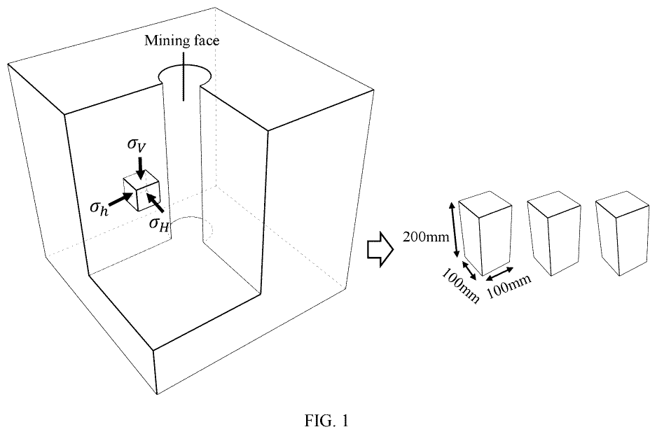

The loaded environment of deep rock mass is determined and standard rock samples are prepared by in-situ sampling. The concrete implementation steps include: as shown in FIG. 1, the rock mass in the construction section of deep engineering is selected as the research object, and the loaded environment of the rock mass in the construction section is obtained by using the in-situ stress measurement method of deep borehole hydraulic fracturing, and the loading levels in the vertical and two horizontal directions are recorded as σ_{V}, σ_{H} and σₕ respectively.

Further, the test standard rock samples are obtained by drilling sampling and laboratory processing, and the length×width×height of the processed standard rock samples is 100 mm×100 mm×200 mm to reflect the spatial distribution of rock pores.

S2, marking the standard rock sample to obtain a test standard rock sample;

Standard rock samples are marked with C-14 by isotope vacuum saturation tracer. The specific implementation steps include:
\textcircled{1} cleaning the standard rock sample with deionized water to remove surface impurities for later use;
\textcircled{2} carrying out isotope labeling of standard rock samples with isotope vacuum saturation tracer.

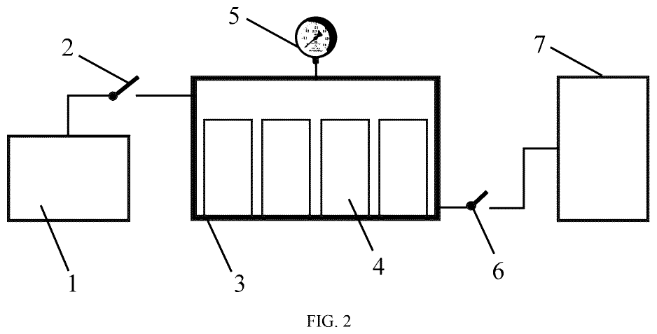

As an additional embodiment, this embodiment provides an isotope vacuum saturation tracer device, as shown in FIG. 2. The test device consists of a vacuum pump 1, a valve A 2, a saturation cylinder 3, a standard rock sample 4, a pressure gauge 5, a valve B 6 and a water injection tank 7, where the vacuum pump 1 is connected with the valve A 2, the valve A 2 is connected with the saturation cylinder 3, the saturation cylinder 3 is used for placing the standard rock sample 4 and is respectively connected with the pressure gauge 5 and the valve B 6, and the valve B 6 is connected with the water injection tank 7, and the water injection tank 7 is filled with clean water with C-14 isotope labelings.

The specific operation flow of the test is as follows: opening the saturation cylinder 3, putting the standard rock sample 4 after impurity treatment in the saturation cylinder 3, and leaving a certain gap between the standard rock samples 4 to ensure that the rock sample is fully saturated, and closing the saturation cylinder 3 to ensure sealing after placing; opening the valve A 2 and closing the valve B 6, turning on the vacuum pump 1 and observing the pressure gauge 5 to ensure that the pressure gauge 5 indicates 0.4 MPa for 4 hours; when meeting the requirements, opening the valve B 6; at this time, the clean water containing C-14 isotope label in the water injection tank 7 will flow into the saturation cylinder 3 until the standard rock sample 4 is submerged, closing the valve B 6, and continue to keep the pressure gauge 5 indicating 0.4 MPa for 4 hours until the standard rock sample 4 is observed without air bubbles overflowing; turning off the vacuum pump 1 and the valve A2, and standing for 4 hours before taking out the standard rock sample 4.
\textcircled{3} Curing and drying the labeled standard rock samples to make the C-14 isotope distribute evenly and stably.

The test standard rock sample labeled with C-14 isotope may be obtained by the method described in S2 \textcircled{1}-\textcircled{3}.

S3, carrying out a loaded catastrophe test on the test standard rock sample to obtain the mining process and test data of the deep rock mass.

As an additional embodiment, this embodiment combines the data-physical fusion method, and the isotope rock mass obtained in S2 captures the point cloud data in S3 by physical technology, and then coordinates are located based on the geometric mathematical relationship.

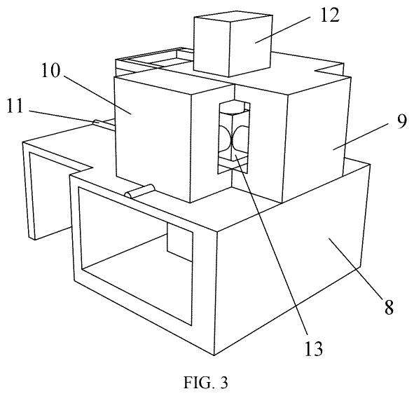

Loaded catastrophe test of standard rock samples are carried out to simulate the mining process of deep rock mass and receive the target test data. The specific implementation steps include: according to the loaded environment of deep rock mass and the features of mining disaster, this embodiment provides a dynamic evolution tracking loading device of pore topology configuration under the loaded condition of deep rock mass, as shown in FIG. 3-FIG. 6. The main structure of the test loading device is shown in FIG. 3, including a supporting base 8, a first horizontal loading device 9, a circular guide rail 11, a vertical loading device 12 and a vertical loading oil cylinder 13.

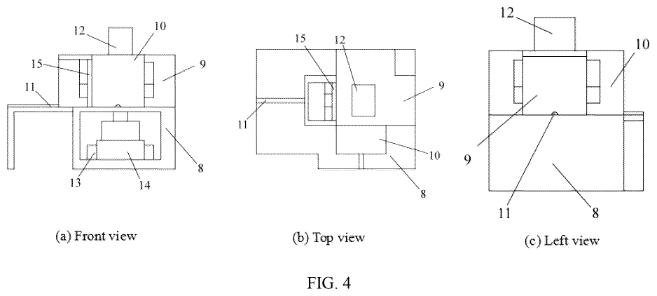

Specifically, the third view of the test loading device is shown in FIG. 4, including a supporting base 8, a first horizontal loading device 9, a circular guide rail 11, a vertical loading device 12, a vertical loading oil cylinder 13, a vertical base 14 and a vertical moving supporting device 15.

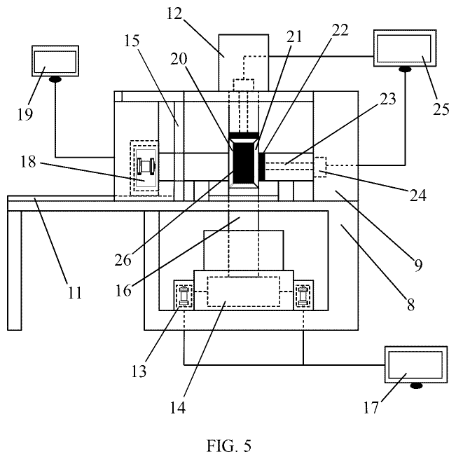

Specifically, the structure of the test device is shown in FIG. 5, including: supporting base 8, first horizontal loading device 9, circular guide rail 11, vertical loading device 12, vertical loading oil cylinder 13, vertical base 14, vertical moving supporting device 15, axial loading cylinder 16, vertical loading control system 17, horizontal loading oil cylinder 18, horizontal loading control system 19, graphite pad 20, copper pad 21, scintillation liquid 22, photomultiplier tube 23, pulse counter 24, isotope detection data tracking system 25 and test standard rock sample 26.

Further, the test standard rock sample 26 has 6 faces, of which the left, front and lower faces are respectively surrounded by the graphite pads 20, and the right, upper and rear faces are respectively surrounded by the copper pads 21. The C-14 isotope in the test standard rock sample 26 will decay and release particles 30 during the test. The graphite pad 20 prevents the β particles 30 from spreading and the β particles may pass through the copper pad 21.

Specifically, the six corresponding graphite pads 20 and copper pads 21 of the test standard rock sample 26 are respectively connected with the axial loading cylinders 16 in six directions, where the axial loading cylinders 16 in the left and right directions are connected with the first horizontal loading device 9, the axial loading cylinders 16 in the front and back directions are connected with the second horizontal loading device 10, and the axial loading cylinders 16 in the up and down directions are connected with the vertical loading device 12, and the first horizontal loading device 9.

Further, the first horizontal loading device 9 and the second horizontal loading device 10 contain the horizontal loading oil cylinder 18, the vertical loading device 12 contains the vertical loading oil cylinder 13, and the horizontal loading control system 19 is connected with the first horizontal loading device 9 and the second horizontal loading device 10. The horizontal loading control system 19 may control the horizontal loading cylinders 18 inside the first horizontal loading device 9 and the second horizontal loading device 10 to apply horizontal stresses OH and on respectively. The vertical loading control system 17 is connected with the vertical loading device 12 and the vertical loading device 12 is located on the vertical base 14. The vertical loading control system 17 may control the vertical loading oil cylinders 13 inside the vertical loading device 12 to apply the vertical stress of Oy, and the real loaded environment for deep rock mass may be simulated through the operation.

Specifically, the first horizontal loading device 9, the second horizontal loading device 10 and the vertical loading device 12 all appear in pairs and act on six faces of the test standard rock sample 26 respectively, and the circular guide rails 11 are arranged at the bottoms of the left first horizontal loading device 9 and the front second horizontal loading device 10, so that the first horizontal loading device 9 and the second horizontal loading device 10 may move on the circular guide rails 11. A vertical moving supporting device 15 is installed in the left first horizontal loading device 9 to support the upper vertical loading device 12. When the left first horizontal loading device 9 moves, the vertical moving supporting device 15 may also move, which is convenient for the installation and disassembly of the test standard rock sample 26.

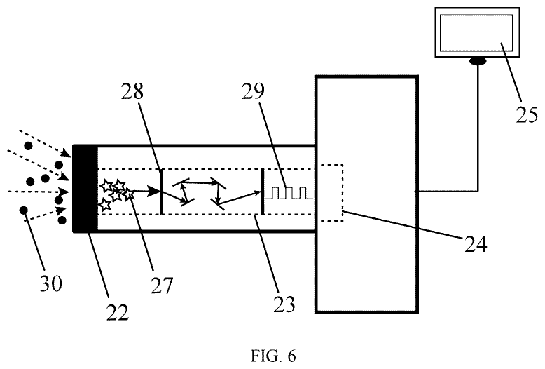

As an additional embodiment, the right first horizontal loading device 9, the rear second horizontal loading device 10 and the axial loading cylinder 16 of the upper vertical loading device 12 contain isotope detecting and tracking devices, which are composed of scintillation liquid 22, photomultiplier tube 23, pulse counter 24, isotope detection data tracking system 25, fluorescent particles 27, photocathode 28, pulse signal 29 and β particles 30 as shown in FIG. 6.

As an additional embodiment, the working principle of the isotope detecting and tracking device is as follows: the interior of the test standard rock sample 26 is uniformly labeled with C-14 isotope through S2, and during the shearing test, the C-14 isotope in the test standard rock sample 26 will decay to generate β particles 30, and the β particles 30 enter the scintillation liquid 22 through the copper pads 21 and interact with each other to form fluorescent particles 27. The fluorescent particles 27 enter the photomultiplier tube 23 through the photocathode 28, and after amplification and processing, they are converted into pulse signals 29. The radiation intensity may be recorded and analyzed in the pulse counter 24, and the results are finally displayed in the isotope detection data tracking system 25.

Further, the implementation process of dynamic evolution tracking loading of pore topology configuration under the loaded condition of deep rock mass is as follows: before the test starts, moving the left first horizontal loading device 9 and the front second horizontal loading device 10 to the distance through the circular guide rail 11, placing the test standard rock sample 26 at the instrument loading position, and then pushing the left first horizontal loading device 9 and the front second horizontal loading device 10 to seal the test standard rock sample 26. At the beginning of the test, the horizontal loading control system 19 controls the first horizontal loading device 9 and the second horizontal loading device 10 to simultaneously apply initial horizontal stress of OH and on to the test standard rock sample 26, and the vertical loading control system 17 controls the initial vertical stress of Oy applied by the vertical loading device 12 to the test standard rock sample 26, and the initial horizontal stress and vertical stress are simultaneously applied. After the initial horizontal stress and vertical stress are applied, it is allowed to stand for 30 min, and the initial isotope radiation data of the test standard rock sample 26 is recorded by the isotope detection data tracking system 25. The vertical loading control system 17 controls the vertical loading device 12 to gradually increase the vertical stress to the standard rock sample 26 until the standard rock sample 26 is damaged by load. The isotope detection data tracking system 25 continuously collects the radiation data of the test standard rock sample 26 during the test. At the end of the test, the horizontal loading control system 19 controls the first horizontal loading device 9 and the second horizontal loading device 10 to unload the horizontal stress to the test standard rock sample 26 respectively. The vertical loading device 12 is controlled by the vertical loading control system 17 to unload the vertical stress to the test standard rock sample 26, and the damaged test standard rock sample 26 is taken out and the test data is saved, thus ending the test.

S4, obtaining point cloud information of the loading process of the test standard rock sample according to the mining process and the test data of the deep rock mass; carrying out three-dimensional reconstruction on the test standard rock sample according to the point cloud data to obtain the spatial dynamic evolution of the loaded behavior of the deep rock mass.

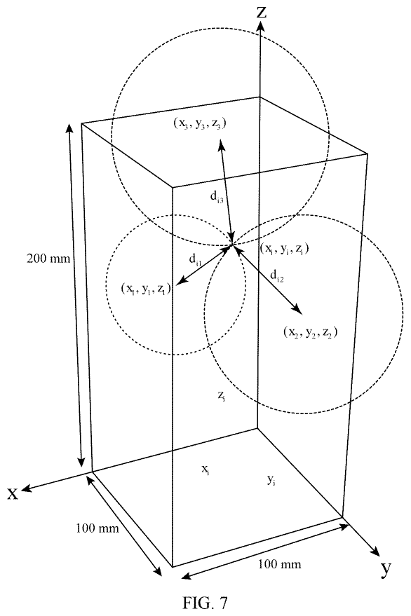

As an additional embodiment, the point cloud information of standard rock samples during the loading process is obtained in real time, and the synchronous reconstruction of three-dimensional loaded rock mass and the real-time tracking of pore topology evolution are realized by solving Poisson equation. The specific implementation steps include: uniformly distributing the same radioactive source intensity in the test standard rock sample obtained by the second step, and measuring different radiation intensities at any position inside the test standard rock sample under the condition that the radioactive source intensity is unchanged. According to the radiation intensity measured at different positions in the test described in S3, the spatial point cloud coordinates at any position in the test standard rock sample are calculated back, and the realization principle is shown in FIG. 7. According to the size data (100 mm×100 mm×200 mm) of the test standard rock sample described in S3, the spatial coordinates of the three isotope detecting and tracking devices may be obtained as (x₁, y₁, z₁)=(50, 0, 100), (x₂, y₂, z₂)=(0, 50, 100), (x₃, y₂, z₃)=(50, 50, 200).

As an additional embodiment, the spatial coordinates of any point in the test standard rock sample 26 are set as (xᵢ, yᵢ, zᵢ), and the radioactive source intensity of this point is Iᵢ₀. During the test, the radiation intensity of this point was captured by three isotope detecting and tracking devices, which are respectively denoted as I₁, I₂ and I₃. According to the relationship between signal intensity and distance of isotope decay, obtaining:

$${I}_{1} = \frac{{I}_{i 0}}{{d}_{i 1}^{2}} , {I}_{2} = \frac{{I}_{i 0}}{{d}_{i 2}^{2}} , {I}_{3} = \frac{{I}_{i 0}}{{d}_{i 3}^{2}}$$

where: Iᵢ₀ is the intensity of the radioactive source at coordinates (xᵢ, yᵢ, zᵢ), dᵢ₁, dᵢ₂ and dᵢ₃ are the distances between the three isotope detectors and the radioactive source respectively, and I₁, I₂ and I₃ are the radiation intensities measured by the radioactive source at distances dᵢ₁, dᵢ₂ and dᵢ₃ respectively.

Further, the distances between the spatial coordinates (xᵢ, yᵢ, zᵢ) and the three detectors may be obtained by the above formula as follows:

$${d}_{i 1} = \sqrt{\frac{{I}_{i 0}}{{I}_{1}}} , {d}_{i 2} = \sqrt{\frac{{I}_{i 0}}{{I}_{2}}} , {d}_{i 3} = \sqrt{\frac{{I}_{i 0}}{{I}_{3}}}$$

Further, according to the spatial geometric relationship, the distances between the spatial coordinates (xᵢ, yᵢ, zᵢ) and the three detectors may be expressed as:

$$\{ \begin{matrix}{d}_{i 1} = \sqrt{{( {x}_{1} - {x}_{i} )}^{2} + {( {y}_{1} - {y}_{i} )}^{2} + {( {z}_{1} - {z}_{i} )}^{2}} \\ {d}_{i 2} = \sqrt{{( {x}_{2} - {x}_{i} )}^{2} + {( {y}_{2} - {y}_{i} )}^{2} + {( {z}_{2} - {z}_{i} )}^{2}} \\ {d}_{i 3} = \sqrt{{( {x}_{3} - {x}_{i} )}^{2} + {( {y}_{3} - {y}_{i} )}^{2} + {( {z}_{3} - {z}_{i} )}^{2}}\end{matrix}$$

where: (x₁, y₁, z₁), (x₂, y₂, z₂) and (x₃, y₂, z₃) are the spatial coordinates of the known isotope detecting and tracking device, and (xᵢ, yᵢ, zᵢ) are the spatial coordinates of any point inside the test standard rock sample.

By combining the above formulas, the spatial coordinates of any point in the test standard rock sample may be calculated as (xᵢ, yᵢ, zᵢ). The test standard rock sample is regarded as composed of n spatial points, and the spatial point cloud data information of the test standard rock sample may be obtained in real time according to the real-time radiation intensity data capture during the test.

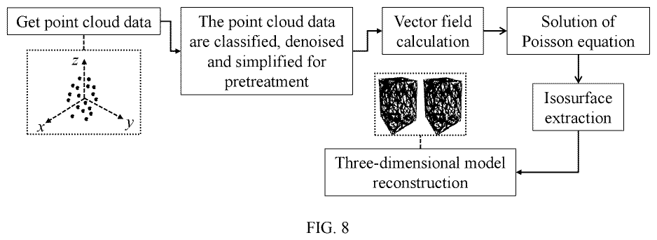

As an additional embodiment, the three-dimensional model of the test standard rock sample is reconstructed by using the spatial point cloud data information through geometric mathematical relations, and its implementation process is shown in FIG. 8, and the specific process is as follows:
(1) based on the above method, the coordinate data of n spatial point clouds in the test rock sample are obtained and recorded as P(xᵢ, yᵢ, zᵢ), i=1, 2, 3 . . . n;
(2) the point cloud data of the test rock sample is pre-processed, including classifying, denoising and simplifying the point cloud data. According to the features of point cloud density and arrangement, the three-dimensional point cloud data during the loading process of rock samples are divided into scattered point clouds.

Furthermore, the original point cloud data is denoised based on statistical methods. The

$$P ( {x}_{i}^{′} , {y}_{i}^{′} , {z}_{i}^{′} ) , i = 1 , 2 , 3 ⋯ n$$

is obtained by standardizing the three-dimensional point cloud data coordinates P(xᵢ, yᵢ, zᵢ), i=1, 2, 3 . . . n of rock samples, and the calculation method is as follows:

$${x}_{i}^{′} = \frac{{x}_{i} - {μ}_{x}}{{σ}_{x}} , {y}_{i}^{′} = \frac{{y}_{i} - {μ}_{y}}{{σ}_{y}} , {z}_{i}^{′} = \frac{{z}_{i} - {μ}_{z}}{{σ}_{z}}$$

where: (xᵢ, yᵢ, zᵢ) are the spatial coordinates of any point inside the test standard rock sample,

$$( {x}_{i}^{′} , {y}_{i}^{′} , {z}_{i}^{′} )$$

are the coordinates after standardization, μₓ, μ_{y} and μ_{z} are the mean values in three coordinate directions respectively, and σₓ, σ_{y} and σ_{z} are the standard deviations in three coordinate directions respectively.

Furthermore, k nearest neighbor search is used to calculate the nearest k points Mₖ(P)={P₁, P₂ . . . Pₖ} in the neighborhood of

$$P ( {x}_{i}^{′} , {y}_{i}^{′} , {z}_{i}^{′} ) ,$$

and kernel density estimation (KDE) is used to calculate the local ρ(P) of each point. According to the local density ρ(P), a threshold ρₜₕ is set, and the points below this threshold are regarded as noise, and the low-density points are removed from the point cloud to obtain denoised point cloud data P_{clean}.

Furthermore, the denoised point cloud data P_{clean} is simplified to improve the efficiency of three-dimensional reconstruction of point clouds of rock samples. According to the point cloud coordinate data information P_{clean}, eight vertex coordinates of the minimum bounding box are determined, the minimum bounding box is divided into small grids, the gravity centers of all contained points in the small grid area are calculated, the distance Di between points in each small grid area is calculated by using the spatial Euclidean distance, and the gravity center points in the small grid area are determined to replace all points in the area to simplify the point cloud data of the original rock mass sample. The preprocessed three-dimensional point cloud data is recorded as

$${P}_{new} ( {x}_{i}^{′} , {y}_{i}^{′} , {z}_{i}^{′} ) .$$

(3) The

$${P}_{new} ( {x}_{i}^{′} , {y}_{i}^{′} , {z}_{i}^{′} )$$

vector field of point cloud data is calculated and Poisson equation is constructed, and finally the three-dimensional model reconstruction of rock sample is completed by solving Poisson equation and extracting isosurface. The nearest k points Mₖ(P_{new})={P_{new1}, P_{new2} . . . P_{newk}} in the

$${P}_{new} ( {x}_{i}^{′} , {y}_{i}^{′} , {z}_{i}^{′} )$$

neighborhood are calculated by k nearest neighbor search, and the mean value P̅_̅{̅n̅e̅w̅}̅ and covariance matrix C of the neighborhood points are calculated according to the coordinate information of the neighborhood points. The eigenvalues λ₁, λ₂, λ₃ and the corresponding eigenvectors \vec{v₁}, \vec{v₂} and \vec{v₃} are obtained by eigenvalue decomposition of the covariance matrix C. The smallest feature vector \vec{v₃} is selected as the normal vector nᵢ and the normal vector is normalized. The calculation method is as follows:

$$= \frac{{n}_{i}}{\vert {n}_{i} \vert}$$

where [symbol] is the normalized normal vector, nᵢ is the normal vector, and |nᵢ| is the module length of the normal vector.

Furthermore, the Poisson equation ∇·(∇φ)=∇· [symbol] , is constructed, and the Poisson equation is discretized by the finite difference method to obtain the scalar field φ. The discretized equation is rewritten into the form of linear equations Aφ=b, and the scalar field φ may be obtained by solving the equations.

Where: ∇ represents divergence operation, φ is scalar field, \hat{n}ᵢ is normalized normal vector, A is coefficient matrix, and b is source term vector.

Furthermore, Marching Cubes method is used to extract isosurface from scalar field φ. The three-dimensional space is divided into cubic grids of N×N×N, each cube is set as a voxel with 8 vertices, the scalar value Sᵢ=φ(Vᵢ), i=0, 1, 2 . . . 7 of each cube is calculated, an index is constructed according to the relationship between the scalar value Sᵢ and the equivalent face value T, linear interpolation is performed between the two vertices to determine the precise position of the triangle, and the triangles generated from all voxels are combined into a complete three-dimensional grid.

Where: Sᵢ is the scalar value of each cube vertex, Vᵢ is the vertex of each cube, and T is the equivalent face value.

Because only the rock sample is labeled with isotopes, but the internal pores of the rock sample are not labeled with isotopes, the radiation intensity of the internal pores of the rock sample is small during the test, which is embodied in the fact that the radiation intensity at the coordinates of the pore position is zero, and the radiation intensity at the coordinates of the solid part of the rock sample exists. According to the radiation intensity value, the real-time observation of the internal solid parts of rock samples and their pore topology configurations may be distinguished.

Real-time three-dimensional reconstruction of rock mass loading process and dynamic tracking of pore topology configuration may be realized through the method of obtaining three-dimensional point cloud data of rock mass and (1)-(3) described in S4.

S5, according to the spatial dynamic evolution, extracting features of the pore topology configuration of the deep loaded rock mass to obtain dynamic evolution features.

Based on image processing technology and fully convolutional neural network method, the pore topology configuration features of loaded rock mass are refined extracted and intelligently deduced. The concrete implementation steps include: dividing the three-dimensional reconstruction model of the test rock sample into m two-dimensional image samples of 100×100 mm along the height direction to form a two-dimensional sample image library {1, 2, 3, . . . , m} of the three-dimensional reconstruction model of a single rock sample. There are solid parts and pore structure parts of rock samples in the two-dimensional image, and the two-dimensional updated sample image database {1′, 2′, 3′, . . . , m′} is obtained by binarization threshold segmentation of the two-dimensional sample image.

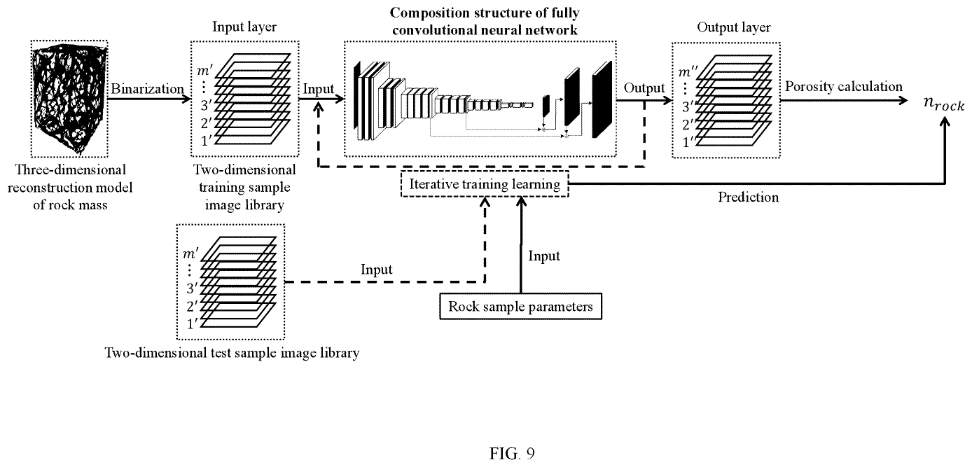

Further, the fully convolutional neural network is used to extract the pore topology configuration features and intelligently deduce the spatial evolution of the two-dimensional updated sample image database {1′, 2′, 3′, . . . , m′}, and the implementation process is shown in FIG. 9. The two-dimensional updated sample image library {1′, 2′, 3′, . . . , m′} is divided into two-dimensional training sample image library and two-dimensional test sample image library, and the two-dimensional training sample image library is used as the input layer to input into the convolution layer of fully convolutional neural network. The pore features of rock mass are extracted by convolution with multiple convolution kernels. The convolution operation is calculated as follows:

$$( f * g ) ( i , j ) = {\sum }_{m = 1}^{M} {\sum }_{n = 1}^{N} f ( m , n ) g ( i - m , j - n )$$

where: f is the input feature map, g is the convolution kernel, (i, j) are the position coordinates in the output feature map, m and n are the row and column indexes when the convolution kernel slides on the input feature map, and M and N are the height and width of the convolution kernel.

Further, the dimension of the feature map is reduced by the pool layer, and the pore distribution features of the image are preserved. The pool operation formula is:

$$MaxPool ( f ) = {max}_{( m , n ) ∈ R} f ( m , n )$$

where: f is the input feature map, R is the area of the pooled window, and m and n are the row and column indexes when the convolution kernel slides on the input feature map.

Further, transposed convolution is used to sample the feature map to the original input size, and the formula of transposed convolution operation is:

$$UpSample ( f ) ( i , j ) = {\sum }_{m , n} f ( m , n ) g ( i - m , j - n )$$

where f is the input feature map, g is the convolution kernel, (i, j) is the position coordinate in the output feature map, and m and n are the row and column indexes when the convolution kernel slides on the input feature map.

Further, the softmax activation function is used for classification as the output of the last convolution layer, and the formula for the operation of the output layer is:

$$\hat{y} = \frac{{e}^{f ( x )}}{{\sum }_{k} {e}^{{f}_{k} ( x )}}$$

where ŷ is the output probability, f(x) is the original output score of the network, and k is the category index.

Through the above operations, the porosity distribution and spatial dynamic evolution features of rock samples may be extracted in real time, and finally the two-dimensional training sample image library {1″, 2″, 3″, . . . , m″} after convolution is output. The pore topology configuration and porosity value of a single two-dimensional image of the test rock sample are obtained through {1″, 2″, 3″, . . . , m″}, and the porosity calculation method is as follows:

$${n}_{rock} = \frac{{S}_{pore}}{{S}_{pore} + {S}_{entity}}$$

where n_{rock} is the porosity of the test rock mass, Sₚₒᵣₑ is the pore area of the rock mass, and S_{entity} is the area of the solid part of the rock mass.

Furthermore, the two-dimensional test sample image library is input into the fully convolutional neural network, so that the fully convolutional neural network may be trained and learned repeatedly to improve the prediction level of the fully convolutional neural network model. The parameters of the rock sample are input into the trained fully convolutional neural network model, including the basic physical and mechanical parameters of the rock test and the in-situ stress levels σ_{V}, σ_{H} and σₕ of the rock sample obtained in the S1, and the trained model is used to intelligently deduce the evolution of the pore topology configuration during the loading process of the rock sample and output the distribution of the pore topology configuration.

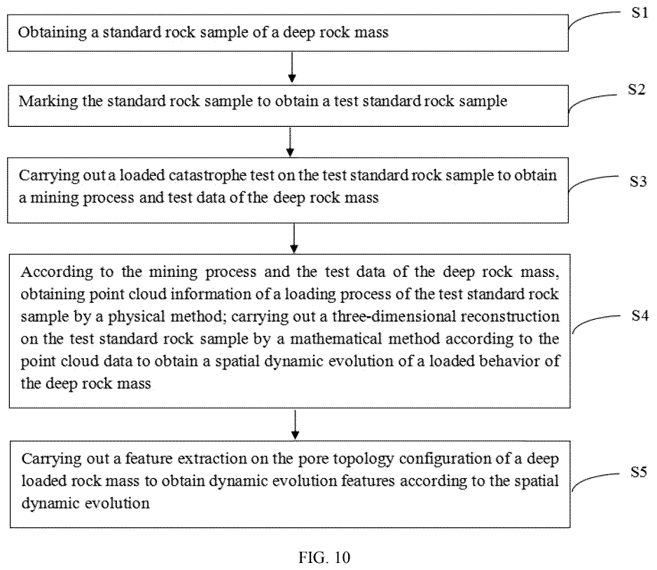

The implementation steps of the method in this embodiment are shown in FIG. 10.

Through the above steps, intelligent deduction based on the evolution tracking test of pore topology configuration of deep loaded rock mass may be realized.

The above is only the preferred embodiment of this application, but the protection scope of this application is not limited to this. Any change or replacement that may be easily thought of by a person familiar with this technical field within the technical scope disclosed in this application should be included in the protection scope of this application. Therefore, the protection scope of this application should be based on the protection scope of the claims.

What is claimed is:

1. An intelligent deduction method of data-physical fusion of pore topology configuration of deep rock mass based on experiments, comprising following steps:
obtaining a standard rock sample of a deep rock mass;
marking the standard rock sample to obtain a test standard rock sample;
carrying out a loaded catastrophe test on the test standard rock sample to obtain a mining process and test data of the deep rock mass;
according to the mining process and the test data of the deep rock mass, obtaining point cloud information of a loading process of the test standard rock sample by a physical method; carrying out a three-dimensional reconstruction on the test standard rock sample by a mathematical method according to the point cloud data to obtain a spatial dynamic evolution of a loaded behavior of the deep rock mass; and
carrying out a feature extraction on the pore topology configuration of a deep loaded rock mass to obtain dynamic evolution features according to the spatial dynamic evolution.

2. The intelligent deduction method of data-physical fusion of pore topology configuration of deep rock mass based on experiments according to claim 1, wherein marking the standard rock sample comprises:
using an isotope vacuum saturation tracer device to mark the standard rock sample with C-14;
wherein the isotope vacuum saturation tracer device comprises a vacuum pump, a valve A, a saturation cylinder, the standard rock sample, a pressure gauge, a valve B and a water injection tank.

3. The intelligent deduction method of data-physical fusion of pore topology configuration of deep rock mass based on experiments according to claim 1, wherein carrying out the loaded catastrophe test on the test standard rock sample comprises:
carrying out the loaded catastrophe test on the test standard rock sample by adopting a dynamic evolution tracking loading device; wherein the dynamic evolution tracking loading device comprises a supporting base, a first horizontal loading device, a second horizontal loading device, a circular guide rail, a vertical loading device and a vertical loading oil cylinder.

4. The intelligent deduction method of data-physical fusion of pore topology configuration of deep rock mass based on experiments according to claim 2, wherein obtaining the point cloud information of the loading process of the test standard rock sample comprises:
according to the mining process and the test data of the deep rock mass, obtaining a spatial distance between the test standard rock sample and an isotope detecting and tracking device;
calculating spatial coordinates of the test standard rock sample according to the spatial distance; and
according to the spatial coordinates, obtaining spatial point cloud data of the loading process of the test standard rock sample.

5. The intelligent deduction method of data-physical fusion of pore topology configuration of deep rock mass based on experiments based on experiments according to claim 1, wherein carrying out the feature extraction on the pore topology configuration of the deep loaded rock mass comprises:
obtaining a two-dimensional sample image of the test standard rock sample according to the spatial dynamic evolution; and
using a fully convolutional neural network to carry out the feature extraction of the pore topology configuration on the two-dimensional sample image to obtain the dynamic evolution features.

6. An intelligent deduction system of data-physical fusion of pore topology configuration of deep rock mass based on experiments, wherein the system comprises:
a rock sample acquisition module, used for obtaining a standard rock sample of a deep rock mass;
a rock sample marking module, used for marking the standard rock sample to obtain a test standard rock sample;
a rock sample test module, used for carrying out a loaded catastrophe test on the test standard rock sample to obtain a mining process and test data of the deep rock mass;
a three-dimensional reconstruction module, used for obtaining point cloud information of a loading process of the test standard rock sample according to the mining process and the test data of the deep rock mass; carrying out a three-dimensional reconstruction on the test standard rock sample according to point cloud data to obtain a spatial dynamic evolution of a loaded behavior of the deep rock mass; and
a feature extraction module, used for carrying out a feature extraction on the pore topology configuration of a deep loaded rock mass to obtain dynamic evolution features according to the spatial dynamic evolution.

7. A computer device, comprising a memory, a processor and a computer program stored on the memory, wherein the processor executes the computer program to realize the steps of the method according to claim 1.
